(19) United States
(12) Patent Application Publication
(10) Pub. No.: US 2022/0273585 A1
(43) Pub. Date: Sep. 1, 2022

(54) Composition comprising at least one natural or synthetic non-euphoric cannabinoid and its method of manufacture

(71) Applicants: Eleonor, Waterloo (BE); Tilman, Baillonville (BE)

(72) Inventors: Cristina LOIRA-PASTORIZA, Bruxelles (BE); Mayéli PRIAN, Rendeux (BE); Fabian PRIEM, Waterloo (BE); Yvan DIERCKXSENS, Overijse (BE)

(21) Appl. No.: 17/679,802

(22) Filed: Feb. 24, 2022

(30) Foreign Application Priority Data
Feb. 26, 2021 (BE) BE2021/5139

(51) Int. Cl. A61K 31/05; A61K 45/06; A61K 47/42; A61K 47/36; A61K 47/44; A61K 47/46; A61K 47/06

(52) U.S. Cl. CPC A61K 31/05; A61K 45/06; A61K 47/42; A61K 47/36; A61K 47/44; A61K 47/46; A61K 47/06

(57) ABSTRACT

The present invention relates to a composition in the form of a friable/brittle thermoformed extrudate comprising at least one natural or synthetic non-euphoric cannabinoid and at least one natural or synthetic carrier chosen from the group consisting of amino acids, natural or synthetic peptides and polypeptides, natural or synthetic proteins, natural or synthetic saccharides, natural or synthetic lipids, urea, the derivatives thereof and the mixtures thereof.

The present invention relates to a composition comprising at least one natural or synthetic non-euphoric cannabinoid, to the manufacturing method thereof and to the use thereof.

According to the invention, a cannabinoid is a molecule whose basic chemical structure is as follows:

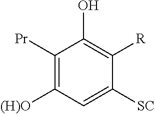

wherein Pr=prenyl group; R=H or COOH; SC (side-chain)=isoprenyl side chain in the para position or alkyl side chain in the para position or aralkyl side chain in the para position.

This chemical structure is presented in the document by Gülck et al. (Trends in Plant Science, 2020, Vol. 25, No. 10) which indicates that the term (phyto-)cannabinoid defines the meroterpenoids with a resorcinyl ring having an isoprenyl, alkyl or aralkyl side chain in the para position.

The terms “non-euphoric cannabinoid”, refer, within the meaning of the present invention, to a cannabinoid which, in an individual/an animal, once absorbed/consumed, does not mainly and significantly alter the mental and/or motor capacities of the individual/of the animal compatible with the activities of daily living, for example giving rise to euphoria, elation, hallucinations, confusion or even any other state affecting the activities of daily living. By way of example, substances causing such states and therefore being euphoric are Δ-9-tetrahydrocannabinol (THC), cocaine, ectasy or even heroin.

Within the meaning of the present invention, the term “natural or synthetic non-euphoric cannabinoid” designates both non-euphoric cannabinoids of natural and/or plant origin and synthetic non-euphoric cannabinoids, but also all derivatives of non-euphoric cannabinoids.

According to the invention, said non-euphoric cannabinoid may in particular be derived from the following plants: Amorpha fruticosa, Cannabis sativa, Cylindrocarponolidum, Glycyrrhizaacanthocarpa, Helichrysumumbraculigerum, Machaeriummultiflorum, Radula spp. (Radula marginata, Radula perrottettii, Radula complanata, . . . ), Rhododendron spp (Rhododendron dauricum, Rhododendron anthopogonoides, . . . ). This list is not exhaustive.

It is recognized that the natural or synthetic non-euphoric cannabinoids are compounds that are very slightly or totally insoluble in water in which they disperse only slightly or not at all, these compounds therefore having very low bioavailability.

However, despite their low bioavailability/bioaccessibility (i.e. despite the small fraction of the administered dose which effectively reaches the blood circulation in unchanged form) but also despite their low solubility and/or despite their low dispersion, in particular in the intestinal environment, the positive effects of the natural or synthetic non-euphoric cannabinoids on various pathologies make them molecules of interest for administration to a human being and/or for veterinary use.

Unfortunately, it appears that the current formulations/compositions comprising natural or synthetic non-euphoric cannabinoids are inappropriate because of their too low or even non-solubility and/or because of their too low or even non-dispersion in aqueous phase and/or because to the low release of these compounds from these formulations/compositions, which ultimately results in a low bioavailability/bioaccessibility of these molecules of interest. It should be noted that it also appears that the current methods of manufacturing such formulations are restrictive and difficult to implement.

The terms “dispersion in aqueous phase (in aqueous medium)”, refer, within the meaning of the present invention, to a system composed of two phases in which one of the two phases, called the dispersed phase, is finely divided in the other, called the dispersant phase. This dispersion can be molecular (solution), colloidal (dispersion of submicronic particles) or coarser (dispersion of particles larger than one μm). More particularly, according to the invention, the terms “dispersion in aqueous phase” designate the suspensions consisting of a solid phase dispersed in an aqueous phase (liquid).

Within the meaning of the present invention, the term “solubility” designates the ability of a substance, called a solute, to dissolve in another substance, called a solvent, to form a homogeneous mixture called a solution.

The object of the invention is to overcome the drawbacks of the state of the art by providing (1) a composition comprising at least one natural or synthetic non-euphoric cannabinoid whose solubility and/or dispersion in aqueous phase (aqueous medium) is increased such that the bioavailability of these compounds is significantly increased and (2) a method for manufacturing such a composition which is easy to implement, which is flexible, which is economically profitable and which ensures that said at least one natural or synthetic non-euphoric cannabinoid is present and homogeneously spread in the final composition obtained.

Moreover, the invention intends to provide a composition which is stable over time, that is to say which retains its properties in terms of solubility and/or dispersion of said at least one natural or synthetic non-euphoric cannabinoid and which retains its properties in terms of the rate of release of this compound over time from a composition (formulation) according to the invention, this in particular in the aqueous phase and more particularly in the intestinal environment.

To at least partially solve these problems, a composition is provided according to the invention, in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibres, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof.

The term “carrier”, refers, within the meaning of the present invention, to a compound or to a molecule, in particular to a compound or to a molecule which allows the dispersion/the solubilization/the integration within it of the active agent (the non-euphoric cannabinoid) so as to form a homogeneous mixture/dispersion.

In particular, according to the invention, said at least one carrier is a thermoplastic carrier, that is to say a carrier having the property of softening when it is heated sufficiently, but which, cooling, becomes again hard, friable/brittle.

According to the invention, said carrier may be a polymer, in particular a thermoplastic polymer.

Within the meaning of the present invention, the term “carrier” designates the molecules/compounds of polymer (for example polysaccharides) or non-polymer (for example amino acids) type.

According to one embodiment, there is therefore provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one amino acid, natural or synthetic peptide and/or polypeptide as a carrier.

By way of example, among the amino acids, the following amino acids may be mentioned: aspartic acid, cysteic acid, muramic acid, pyroglutamic acid, alanine, arginine, asparagine, aspartate, citrulline, cysteine, glutamate, glutamine, glycine, histidine, homocysteine, homoserine, hydroxyproline, isovaline, isoleucine, leucine, lysine, methionine, methylalanine, norleucine, norvaline, ornithine, phenylalanine, proline, sarcosine, serine, threonine, tryptophan, tyrosine, valine, the derivatives thereof and the mixtures thereof.

By way of example, among the peptides and the polypeptides, aspartame, carnosine, the derivatives thereof and the mixtures thereof may be mentioned.

According to one embodiment, there is therefore provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic protein as a carrier selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof.

According to one embodiment, there is therefore provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic saccharide as a carrier selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof.

The term “saccharide” includes monosaccharides, oligosaccharides and polysaccharides.

By way of example, among the soluble or insoluble fibers Konjac fibers, acacia fibers, inulin fibers or even arabinoxylan fibers may be mentioned. This list is not exhaustive.

According to one embodiment, there is also provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic dextrin as a carrier.

By way of example, among the dextrins, pea dextrins, potato dextrins, corn dextrins, the derivatives thereof and the mixtures thereof may be mentioned. This list is not exhaustive.

According to one embodiment, there is also provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic cyclodextrin as a carrier.

By way of example, among the cyclodextrins, α-cyclodextrin, β-cyclodextrin, γ-cyclodextrin, the derivatives thereof and the mixtures thereof may be mentioned. This list is not exhaustive.

According to one embodiment, there is also provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic hyaluronate as a carrier.

By way of example, among the hyaluronates, hyaluronic acid, sodium hyaluronate, the derivatives thereof and the mixtures thereof may be mentioned. This list is not exhaustive.

According to one embodiment, there is also provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic carrageenan as a carrier.

By way of example, among the carrageenans, κ-carrageenan, η-carrageenan, λ-carrageenan, the derivatives thereof and the mixtures thereof may be mentioned. This list is not exhaustive.

According to one embodiment, there is also provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic starch as a carrier.

By way of example, among the starches, corn starch, potato starch, rice starch, wheat starch, buckwheat starch, chestnut starch, sweet potato starch or even cassava starch may be mentioned. This list is not exhaustive.

According to one embodiment, there is therefore provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic lipid as a carrier selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof.

The term “extrudate”, refers, within the meaning of the present invention, to a material which comes out of an extruder, in particular of the die of an extruder.

The terms “thermoformed extrudate”, refer, within the meaning of the present invention, to a material which comes out of an apparatus, in particular which comes out of an extruder, in which it has undergone a transformation by the effect of heat, optionally by the combined effect of heat and of shear forces of a worm screw. Such a transformation by the effect of heat, optinally by the combined effect of heat and of shear forces of a worm screw, can be obtained with the Hot Melt Extrusion, HME, technique.

A thermoformed extrudate according to the invention can therefore be obtained according to the hot melt extrusion technique which allows achieving the molecular dispersion of an active agent (active substance) within a carrier (for example within a polymer/a polymer matrix) to form solid dispersions, the active agent and/or the carrier used undergoing a change of state of the material. The active agent and/or the carrier therefore passes from a first state of the material to a second state of the material, which second state of the material implying that said active agent and/or said carrier is at least partially in amorphous form. This solid dispersion and this change of state of the material are possible thanks to a heat supply and possibly thanks to the stress applied by the movement of the worm screws on the material in an extruder. Finally, the hot melt extrusion results, at the outlet, in the formation of a friable/brittle thermoformed extrudate in the form of a strand which can then in particular be pelletized or crushed.

In particular, a thermoformed extrudate according to the invention is an extrudate in which a non-euphoric cannabinoid and/or at least one natural or synthetic carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibres, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof, has/have been melted to give rise to a solid dispersion consisting of a glassy structure comprising a molecular mixture of said non-euphoric cannabinoid and of said at least one carrier such that said non-euphoric cannabinoid is dispersed within said at least one carrier.

The terms “glassy structure”, refer, within the meaning of the present invention, to a structure comprising/being formed by a molecular mixture of at least two compounds/molecules, at least one of these two compounds/molecules being at least partially in a non-crystalline form, in particular being at least partially in an amorphous form. In particular, in the context of the present invention, such a glassy structure is obtained by a change of state of the material comprising the active ingredient (the non-euphoric cannabinoid) and/or the carrier used, which change of state of the material is obtained by hot melt extrusion, the material being subjected to heating and undergoing melting.

The terms “solid dispersion”, refer, within the meaning of the present invention, to a mixture/a system of at least two compounds/molecules, at least one of these two compounds/molecules being at least partially in a non-crystalline form, in particular being at least partially in an amorphous form.

In particular, a “solid dispersion” consists of a molecular dispersion of an active ingredient/of an active substance at least partially in an amorphous form within a carrier, for example within a polymer matrix.

The terms “friable/brittle thermoformed extrudate”, refer, within the meaning of the present invention, to an extrudate which easily disaggregates, which can be easily reduced to powder and/or to small fragments/pieces. In particular, it is understood, within the meaning of the present invention, an extrudate which does not have elastic and sticky properties and which does not have a gummy texture. A friable/brittle thermoformed extrudate within the meaning of the present invention is therefore not a chewing-gum type extrudate.

According to the invention, the thermoformed extrudate is not in the form of a chewing gum.

In particular, a friable/brittle thermoformed extrudate according to the invention, optionally packaged in pellet, flake, granule, powder, effervescent or non-effervescent tablet, injectable or non-injectable solution, suspension, gel, ointment or even in any other suitable form allowing administration to an animal or to a human being, is an extrudate in which the active ingredient (said at least one natural or synthetic non-euphoric cannabinoid) and/or said at least one carrier is/are melted.

In particular, the composition according to the invention, more particularly the composition in the form of a friable/brittle thermoformed extrudate according to the invention, optionally packaged in pellet, flake, granule, powder, effervescent or non-effervescent tablet, injectable or non-injectable solution, suspension, gel, ointment or even in any other suitable form allowing administration to an animal or to a human being, is a solid dispersion in which said at least one natural or synthetic non-euphoric cannabinoid is dispersed in said at least one carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof.

According to one embodiment, there is therefore provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one amino acid, natural or synthetic peptide and/or at least one polypeptide as a carrier, said composition being a solid dispersion in which said at least one natural or synthetic non-euphoric cannabinoid is dispersed in said at least one amino acid, natural or synthetic at least one peptide and/or at least one polypeptide as a carrier, which carrier is/has been melted.

According to one embodiment, there is therefore provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic protein as a carrier selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, said composition being a solid dispersion in which said at least one natural or synthetic non-euphoric cannabinoid is dispersed in said at least one natural or synthetic protein as a carrier selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, which carrier is/has been melted.

According to one embodiment, there is therefore provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic saccharide as a carrier selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, said composition being a solid dispersion in which said at least one natural or synthetic non-euphoric cannabinoid is dispersed in said at least one natural or synthetic saccharide as a carrier selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, which carrier is/has been melted.

According to one embodiment, there is therefore provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic dextrin as a carrier, said composition being a solid dispersion in which said at least one natural or synthetic non-euphoric cannabinoid is dispersed in said at least one natural or synthetic dextrin as a carrier, which carrier is/has been melted.

According to one embodiment, there is therefore provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic cyclodextrin as a carrier, said composition being a solid dispersion in which said at least one natural or synthetic non-euphoric cannabinoid is dispersed in said at least one natural or synthetic cyclodextrin as a carrier, which carrier is/has been melted.

According to one embodiment, there is therefore provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic hyaluronate as a carrier, said composition being a solid dispersion in which said at least one natural or synthetic non-euphoric cannabinoid is dispersed in said at least one natural or synthetic hyaluronate as a carrier, which carrier is/has been melted.

According to one embodiment, there is therefore provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic carrageenan as a carrier, said composition being a solid dispersion in which said at least one natural or synthetic non-euphoric cannabinoid is dispersed in said at least one natural or synthetic carrageenan as a carrier, which carrier is/has been melted.

According to one embodiment, there is therefore provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic starch and/or at least one modified natural or synthetic starch and/or at least one pregelatinized natural or synthetic starch as a carrier, said composition being a solid dispersion in which said at least one natural or synthetic non-euphoric cannabinoid is dispersed in said at least one natural or synthetic starch and/or in said at least one modified natural or synthetic starch and/or in said at least one pregelatinized natural or synthetic starch as a carrier, which carrier is/has been melted.

According to one embodiment, there is therefore provided according to the invention, a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid and at least one natural or synthetic lipid as a carrier, said composition being a solid dispersion in which said at least one natural or synthetic non-euphoric cannabinoid is dispersed in said at least one natural or synthetic lipid as a carrier selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, which carrier is/has been melted.

A friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, according to the invention is obtained by hot thermoforming, in particular by hot thermoforming using the hot melt extrusion technique. According to the invention, the hot thermoforming therefore relates more particularly to the hot melt extrusion technique.

Advantageously, according to the invention, the hot melt extrusion causes the active agent (the non-euphoric cannabinoid) and the carrier to be (preferably simultaneously) mixed, heated, melted, homogenized and extruded. An intense mixing and a forced stirring, in particular by the worm screws, during the hot melt extrusion method causes the disaggregation of the active particles in the molten carrier, giving rise to a solid dispersion. The material (active agent+carrier) is finally in a “glassy state” or “glassy structure” and behaves like a friable/brittle solid, but without a totally crystalline structure and having a weak arrangement.

There is therefore provided according to the invention a composition obtained by hot thermoforming in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, obtained by hot thermoforming, in particular obtained by hot melt extrusion, said composition comprising at least one natural or synthetic euphoric cannabinoid and at least one natural or synthetic carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof.

A friable/brittle thermoformed extrudate according to the invention, optionally packaged in pellet, flake, granule, powder, effervescent or non-effervescent tablet, injectable or non-injectable solution, suspension, gel, ointment or even in any other suitable form allowing administration to an animal or to a human being, can therefore be obtained according to the hot melt extrusion technique which allows achieving the molecular dispersion of an active agent (active substance) within a carrier, in particular within a polymer matrix (within a polymer), to form solid dispersions. This solid dispersion is possible thanks to a heat supply and possibly thanks to the stress applied by the movement of the worm screws on the material in an extruder. Finally, the hot melt extrusion results, at the outlet, in the formation of a friable/brittle thermoformed extrudate in the form of a strand which can then in particular be pelletized or crushed.

The hot melt extrusion technique can be carried out without the supply of a liquid phase but relies on a heat supply (heating) to ensure a transformation of the material (change of state of the material) by thermoforming. The hot melt extrusion technique gives rise to a transformation of the material (change of state of the material), in particular to a glassy structure obtained under the action of heat (heating), the particles constituting the powders being no longer all present in their crystalline initial (native) form at the end of the hot melt extrusion method but having undergone a transformation by thermoforming.

According to the invention, preferably, said at least one natural or synthetic non-euphoric cannabinoid comprises at least one first amorphous phase and optionally one second crystalline phase.

The terms “comprises at least one first amorphous phase and optionally one second crystalline phase”, mean, within the meaning of the present invention, that said at least one natural or synthetic non-euphoric cannabinoid can either comprise 100% by mass of an amorphous phase, or can simultaneously comprise one first amorphous phase and one second crystalline phase, the sum of the percentages by mass of the first and second phases being in this case equal to 100. In other words, the composition according to the invention can comprise said at least one natural or synthetic non-euphoric cannabinoid (1) totally in amorphous form or (2) partly in amorphous form (phase) and partly in crystalline form (phase).

It should be noted that a phase is called amorphous when the atoms constituting this phase do not respect any order at medium and long distance, which distinguishes it from a so-called crystalline phase.

In the context of the present invention, an analysis by differential scanning calorimetry (DSC) which is well known to those skilled in the art can be used in order to measure the difference in heat exchange between a reference and a studied product. This technique allows determining the melting temperatures, the crystallization temperatures or the phase transition temperatures as well as the reaction enthalpies. In particular, such an analysis allows obtaining curves where the presence of an endothermic peak (heat absorption) shows the melting of the analyzed sample. A melting peak indicates the presence of a crystalline form in the sample. On the contrary, when the melting peak is not present, the material is in the amorphous state.

The composition according to the invention is therefore preferably in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, wherein said at least one natural or synthetic non-euphoric cannabinoid (active ingredient) comprises at least one first amorphous phase and optionally one second crystalline phase, which phase(s) is/are dispersed within at least one carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibres, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof.

The terms “natural amino acid”, refer, within the meaning of the present invention, to any amino acid naturally present in the living world, in particular to a plant or animal amino acid.

The terms “synthetic amino acid”, refer, within the meaning of the present invention, to any amino acid subject to human intervention, in particular to an amino acid obtained from a chemical or biochemical or biotechnological process.

The terms “natural peptide or polypeptide”, refer, within the meaning of the present invention, to any peptide or polypeptide naturally present in the living world, in particular to a plant or animal peptide or polypeptide.

The terms “synthetic peptide or polypeptide”, refer, within the meaning of the present invention, to any peptide or polypeptide subject to human intervention, in particular to a peptide or a polypeptide obtained from a chemical or biochemical or biotechnological process.

The terms “natural protein”, refer, within the meaning of the present invention, to any protein naturally present in the living world, in particular to a plant or animal protein.

The terms “synthetic protein”, refer, within the meaning of the present invention, to any protein subject to human intervention, in particular to a protein obtained from a chemical or biochemical or biotechnological process.

The terms “natural saccharide”, refer, within the meaning of the present invention, to any saccharide naturally present in the living world, in particular to a plant or animal saccharide.

The terms “synthetic saccharide”, refer, within the meaning of the present invention, to any saccharide subject to human intervention, in particular to any saccharide obtained from a chemical or biochemical or biotechnological process.

The terms “saccharide” includes any form of saccharide including monosaccharides, oligosaccharides and polysaccharides.

The terms “natural lipid”, refer, within the meaning of the present invention, to any lipid naturally present in the living world, in particular to a plant lipid.

The terms “synthetic lipid”, refer, within the meaning of the present invention, to any lipid subject to human intervention, in particular to a lipid obtained from a chemical or biochemical or biotechnological process.

The terms “natural alginate”, refer, within the meaning of the present invention, to any alginate naturally present in the living world, in particular to a plant or animal alginate.

The terms “synthetic alginate”, refer, within the meaning of the present invention, to any alginate subject to human intervention, in particular to any alginate obtained from a chemical or biochemical or biotechnological process.

The terms “natural polysaccharide”, refer, within the meaning of the present invention, to any polysaccharide naturally present in the living world, in particular to a plant or animal polysaccharide.

The terms “synthetic polysaccharide”, refer, within the meaning of the present invention, to any polysaccharide subject to human intervention, in particular to any polysaccharide obtained from a chemical or biochemical or biotechnological process.

The terms “natural oligosaccharide”, refer, within the meaning of the present invention, to any oligosaccharide naturally present in the living world, in particular to a plant or animal oligosaccharide.

The terms “synthetic oligosaccharide”, refer, within the meaning of the present invention, to any oligosaccharide subject to human intervention, in particular to any oligosaccharide obtained from a chemical or biochemical or biotechnological process.

The terms “natural dextrin”, refer, within the meaning of the present invention, to any dextrin naturally present in the living world, in particular to a plant or animal dextrin.

The terms “synthetic dextrin”, refer, within the meaning of the present invention, to any dextrin subject to human intervention, in particular to a dextrin obtained from a chemical or biochemical or biotechnological process.

The terms “natural cyclodextrin”, refer, within the meaning of the present invention, to any cyclodextrin naturally present in the living world, in particular to a plant or animal cyclodextrin.

The terms “synthetic cyclodextrin”, refer, within the meaning of the present invention, to any cyclodextrin subject to human intervention, in particular to a cyclodextrin obtained from a chemical or biochemical or biotechnological process.

The terms “natural hyaluronate”, refer, within the meaning of the present invention, to any hyaluronate naturally present in the living world, in particular to a plant or animal hyaluronate.

The terms “synthetic hyaluronate”, refer, within the meaning of the present invention, to any hyaluronate subject to human intervention, in particular to any hyaluronate obtained from a chemical or biochemical or biotechnological process.

The terms “natural carrageenan”, refer, within the meaning of the present invention, to any carrageenan naturally present in the living world, in particular to a plant carrageenan.

The terms “synthetic carrageenan”, refer, within the meaning of the present invention, to any carrageenan subject to human intervention, in particular to a carrageenan obtained from a chemical or biochemical or biotechnological process.

The terms “natural starch”, refer, within the meaning of the present invention, to any starch naturally present in the living world, in particular to a plant or animal starch.

The terms “synthetic starch”, refer, within the meaning of the present invention, to any starch subject to human intervention, in particular to any starch obtained from a chemical or biochemical or biotechnological process.

It has been determined, in the context of the present invention, that such a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprising at least one natural or synthetic non-euphoric cannabinoid as an active ingredient and at least one carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibers, carrageenan, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof, has a significantly higher solubility and/or a dispersion in aqueous phase (aqueous medium) of said at least one natural or synthetic non-euphoric cannabinoid. Moreover, it has been shown that such a composition according to the invention has a significantly increased bioavailability/bioaccessibility of said at least one natural or synthetic non-euphoric cannabinoid relative to the bioavailability/bioaccessibility observed for the current compositions.

According to the invention, the active ingredient, that is to say said at least one natural or synthetic non-euphoric cannabinoid, is dispersed/spread/distributed in a homogeneous manner within at least one carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibres, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof, the active ingredient and/or said at least one carrier being melted during the thermoforming manufacturing method implemented according to the invention and described below.

In addition, a composition according to the invention can be stored for several months without its properties being altered. In particular, it has been demonstrated that a composition according to the invention retains its properties in terms of solubility and/or dispersion in aqueous phase (aqueous medium) of said at least one natural or synthetic non-euphoric cannabinoid and in terms of release rate of this compound over time from a composition/formulation according to the invention, this in particular in the aqueous phase.

Advantageously, according to the invention, said friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, comprises a thermoformed mixture of at least one natural or synthetic non-euphoric cannabinoid and of at least one natural or synthetic carrier chosen from the group consisting of acids amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acids esters, the derivatives thereof and the mixtures thereof, urea, the derivatives thereof and the mixtures thereof.

Alternatively, according to the invention, said friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, consists of a thermoformed mixture of at least one natural or synthetic non-euphoric cannabinoid and of at least one carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibres, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof.

There is therefore provided according to the invention a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, obtained by hot thermoforming, in particular obtained by hot melt extrusion, said friable/brittle thermoformed extrudate comprising a thermoformed mixture of at least one natural or synthetic non-euphoric cannabinoid and of at least one carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibres, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof.

The terms “thermoformed mixture”, refer, within the meaning of the present invention, to a mixture which comes out of an apparatus, in particular which comes out of an extruder, in which it has undergone a transformation by the effect of heat, optionally by the combined effect of heat and of shear forces of a worm screw. Such a transformation by the effect of heat, optionally by the combined effect of heat and of shear forces of a worm screw, can be obtained with the hot melt extrusion technique (HME).

In particular, a thermoformed mixture according to the invention is a mixture in which the active ingredient (said at least one natural or synthetic non-euphoric cannabinoid) and/or said at least one carrier is/are melted/was/were melted.

A thermoformed mixture according to the invention can therefore be obtained according to the hot melt extrusion technique which allows achieving the molecular dispersion of an active agent (active substance) within a carrier, for example within a polymer matrix (within a polymer) to form solid dispersions, the active agent and/or the carrier used undergoing a change of state of the material. The active agent and/or the polymer therefore passes from a first state of the material to a second state of the material, which second state of the material implying that said active agent and/or said carrier is at least partially in amorphous form.

According to the invention, said at least one natural or synthetic non-euphoric cannabinoid comprises one first amorphous phase.

Advantageously, according to the invention, said at least one natural or synthetic non-euphoric cannabinoid predominantly comprises at least one first amorphous phase.

Preferably, according to the invention, said at least one natural or synthetic non-euphoric cannabinoid comprises between 51 and 100% by mass of an amorphous phase and between 0 and 49% of a crystalline phase.

The terms “predominantly at least one first amorphous phase”, mean, within the meaning of the present invention, that said at least one natural or synthetic non-euphoric cannabinoid comprises between 50 and 100% by mass of an amorphous phase and between 0 and 50% of a crystalline phase, more particularly that said at least one natural or synthetic non-euphoric cannabinoid comprises between 51 and 100% by mass of an amorphous phase and between 0 and 49% of a crystalline phase.

Preferably, according to the invention, said at least one natural or synthetic non-euphoric cannabinoid is chosen from the group consisting of cannabidiol (CBD), cannabigerol (CBG), cannabichromene (CBC), cannabidivarin (CBDV), cannabidiolic acid (CBDA), cannabigerovarin (CBGV), cannabichromenic acid (CBCA), cannabiorcichromenic acid, 8-chloro-cannabiorcichromenic acid, cannachromevarinic acid (CBCVA), lacannabielsoin (CBE), cannabicyclol (CBL), perrottetinene, perrottittenic acid, lunularic acid, vittatin, helichrysum CBG, amorfrutins (A, B, C and D), machaeridiols (A, B, C), daurichromenic acid, anthopogocyclolic acid, anthopogochromenic acid, geranyl-orsellinic acid, confluentine, griffolic acid, rubiginosin A, the derivatives thereof and the mixtures thereof.

By way of example, when said at least one natural or synthetic protein is collagen or gelatin, it may be collagen or gelatin of animal origin (fish, pork, beef, . . . ).

In particular and advantageously according to the invention, said collagen hydrolysates are collagen hydrolysates not having/lacking the ability to gel, that is to say non-gelling collagen hydrolysates.

The term “protein” designates a macromolecule consisting of different amino acids that are linked together. The protein is the most common element and the basic molecular unit of any living being. The proteins are characterized by the length of their molecular chain.

The terms “amino acid” designate a carboxylic acid which also has an amine functional group and the term “peptide” designates a polymer of amino acids.

The term “collagen” designates a protein composed of three associated alpha polypeptide chains. These chains are linked by hydrogen bonds between hydroxylysine and hydroxyproline and by covalent bonds.

The term “gelatin” designates collagen which has been modified, the molecular chains preferably remaining sufficiently long to ensure the functional properties desired with gelatin (viscosity, holding of the jelly).

The terms “collagen peptides” designate small ordered groups of amino acids resulting from the fragmentation of the collagen molecule. Depending on the hydrolysis technique used, these groups are more or less important and more or less active.

The terms “hydrolyzed collagen” indicate that the collagen has been hydrolyzed, in particular to make it more assimilable.

Preferentially, according to the invention, said collagens and/or said collagen hydrolysates and/or said gelatins have a molecular weight comprised between 50 and 300000 Da, preferably between 100 and 275000 Da, preferably between 150 and 250000 Da, preferably between 200 and 225000 Da, preferably between 250 and 200000 Da, preferably between 300 and 175000 Da, preferably between 350 and 150000 Da, preferably between 400 and 125000 Da, preferably between 450 and 100000 Da, preferably between 500 and 75000 Da, preferably between 550 and 50000 Da, preferably between 600 and 40000 Da, preferably between 650 and 30000 Da, preferably between 700 and 20000 Da, preferably between 750 and 10000 Da, preferably between 800 and 9000 Da, preferably between 850 and 8000 Da, preferably between 900 and 7000 Da, preferably between 950 and 6000 Da, preferably between 1000 and 5000 Da, preferably between 1050 and 4000 Da, preferably between 1100 and 3000 Da, preferably between 1150 and 2000 Da, preferably between 1200 and 1000 Da.

Advantageously, according to the invention, said collagens and/or said collagen hydrolysates and/or said gelatins have a molecular weight comprised between 1000 and 300000 Da, preferably between 1500 and 150000 Da, preferably between 2000 and 60000 Da.

Preferably, according to the invention, said collagens and/or said collagen hydrolysates have a molecular weight equal to 50 Da or equal to 100 Da or equal to 150 Da or equal to 200 Da or equal to 250 Da or equal to 300 Da or equal to 350 Da or equal to 400 Da or equal to 450 Da or equal to 500 Da or equal to 550 Da or equal to 600 Da or equal to 650 Da or equal to 700 Da or equal to 750 Da or equal to 800 Da or equal to 850 Da or equal to 900 or equal to 950 Da or equal to 1000 Da or equal to 1100 Da or equal to 1200 or equal to 1300 Da or equal to 1400 Da or equal to 1500 Da or equal to 1600 Da or equal to 1700 Da or equal to 1800 Da or equal to 1900 Da or equal to 2000 Da or equal to 2500 Da or equal to 3000 Da or equal to 3500 Da or equal to 4000 Da or equal to 4500 Da or equal to 5000 Da or equal to 5500 Da or equal to 6000 Da or equal to 6500 Da or equal to 7000 Da or equal to 7500 Da or equal to 8000 Da or equal to 8500 Da or equal to 9000 Da or equal to 9500 Da or equal to 10000 Da or equal to 12500 Da or equal to 15000 Da or equal to 17500 Da or equal to 20000 Da or equal to 22500 Da or equal to 25000 Da or equal to 27500 Da or equal to 30000 Da or equal to 32500 Da or equal to 35000 Da or equal to 37500 Da or equal to 40000 Da or equal to 42500 Da or equal to 45000 Da or equal to 47500 Da or equal to 50000 Da or equal to 55000 Da or equal to 60000 Da or equal to 65000 Da or equal to 70000 Da or equal to 75000 Da or equal to 80000 Da or equal to 85000 Da or equal to 90000 Da or equal to 100000 Da or equal to 110000 Da or equal to 120000 Da or equal to 130000 Da or equal to 140000 Da or equal to 150000 Da or equal to 160000 Da or equal to 170000 Da or equal to 180000 Da or equal to 190000 Da or equal to 200000 Da or equal to 210000 Da or equal to 220000 Da or equal to 230000 Da or equal to 240000 Da or equal to 250000 Da or equal to 260000 Da or equal to 270000 Da or equal to 280000 Da or equal to 290000 Da or equal to 300000 Da.

According to one embodiment according to the invention, when said at least one natural or synthetic protein is collagen, the latter has a molecular weight comprised between 900 and 7000 Da, preferably a molecular weight comprised between 950 and 5000 Da, preferentially a molecular weight comprised between 1000 and 3000 Da.

According to one embodiment according to the invention, when said at least one natural or synthetic protein is collagen, the latter has a molecular weight equal to 2000 Da or equal to 3000 Da or equal to 5000 Da or equal to 50000 Da.

According to one embodiment according to the invention, when said at least one natural or synthetic protein is gelatin, the latter has a molecular weight comprised between 900 and 6000 Da, preferably a molecular weight comprised between 950 and 5000 Da, preferentially a molecular weight comprised between 1000 and 3000 Da.

According to one embodiment according to the invention, when said at least one natural or synthetic protein is gelatin, the latter has a molecular weight equal to 2000 Da or equal to 3000 Da or equal to 5000 Da or equal to 50000 Da.

According to one embodiment according to the invention, when said at least one natural or synthetic protein is a hydrolyzed collagen or a collagen peptide, the latter has a molecular weight comprised between 900 and 6000 Da, preferably a molecular weight comprised between 950 and 5000 Da, preferentially a molecular weight comprised between 1000 and 3000 Da.

According to one embodiment according to the invention, when said at least one natural or synthetic protein is a hydrolyzed collagen or a collagen peptide, the latter has a molecular weight equal to 2000 Da or equal to 3000 Da or equal to 5000 Da or equal to 50000 Da.

According to one embodiment, the composition according to the invention further comprises at least one additional natural or synthetic carrier, for example polyvinyl acetate, polyvinylpyrrolidone (PVP), polyvinylpyrrolidone-co-vinyl acetate, polyethylene-co-vinyl acetate, polyvinyl acid co-methacrylic acetate, polyethylene oxide, polylactide-co-glycolide, polyvinyl alcohol, polycarbophil, polycaprolactone, ethylene-vinyl copolymer, the derivatives thereof and the mixtures thereof.

Advantageously, the composition according to the invention also comprises at least one plasticizer. The addition of a plasticizer to a composition according to the invention allows obtaining a composition according to the invention through a manufacturing method where temperatures below the melting points of said at least one natural or synthetic non-euphoric cannabinoid and of the carrier can be used in order to guarantee all the same a melting of these two compounds and the dispersion/spread/distribution of said at least one natural or synthetic non-euphoric cannabinoid within the carrier.

Preferentially, according to the invention, said plasticizer is chosen from the group consisting of polyols, lipids, lecithins, sucrose esters, water, triethyl citrate, polyethylene glycol, glycerol, dibutyl sebate, butyl stearate, glycerol monostearate, sodium dodecyl sulphate, diethyl phthalate, the derivatives thereof and the mixtures thereof.

According to the invention, the preferred plasticizers are glycerol, water, polyethylene glycol and triethyl citrate.

Advantageously, according to the invention, said at least one non-euphoric cannabinoid is present in an amount of 1% to 80% by weight based on the total weight of the composition, preferably in an amount of 10% to 60% by weight based on the total weight of the composition, preferably in an amount of 15% to 50% by weight based on the total weight of the composition, preferentially in an amount of 20% to 40% by weight based on the total weight of the composition.

Preferentially, according to the invention, said at least one non-euphoric cannabinoid is present in an amount of 20% by weight based on the total weight of the composition, or in an amount of 25% by weight based on the total weight of the composition, or in an amount of 30% by weight based on the total weight of the composition, or in an amount of 35% by weight based on the total weight of the composition, or in an amount of 40% by weight based on the total weight of the composition.

Preferably, according to the invention, said at least one natural or synthetic carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof, is present in an amount of 5% to 90% by weight based on the total weight of the composition, preferably in an amount of 15% to 85% by weight based on the total weight of the composition, preferentially in an amount of 20% to 80% by weight based on the total weight of the composition, more preferentially in an amount of 50% to 75% by weight based on the total weight of the composition.

Advantageously, according to the invention, said at least one natural or synthetic carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof, is present in an amount of 5% by weight based on the total weight of the composition, or in an amount of 10% by weight based on the total weight of the composition, or in an amount of 15% by weight based on the total weight of the composition, or in an amount of 20% by weight based on the total weight of the composition, or in an amount of 25% by weight based on the total weight of the composition, or in an amount of 30% by weight based on the total weight of the composition, or in an amount of 35% by weight based on the total weight of the composition, or in an amount of 40% by weight based on the weight total of the composition, or in an amount of 45% by weight based on the total weight of the composition, or in an amount of 50% by weight based on the total weight of the composition, or in an amount of 55% by weight based on the total weight of the composition, or in an amount of 60% by weight based on the total weight of the composition, or in an amount of 65% by weight based on the total weight of the composition, or in an amount of 70% by weight based on the total weight of the composition, or in an amount of 75% by weight based on the total weight of the composition.

When it is a mixture of two carriers according to the invention, the ratio between these two carriers is preferentially of about 10:90 or of about 20:80 or of about 30:70 or of about 40:60 or of about 50:50.

Advantageously, according to the invention, said at least one plasticizer is present in an amount of 5% to 30% by weight based on the total weight of the composition, preferably in an amount of 10% to 25% by weight based on the weight total of the composition, preferentially in an amount of 15% to 20% by weight based on the total weight of the composition.

Advantageously, according to the invention, said at least one plasticizer is present in an amount of 5% by weight based on the total weight of the composition, or in an amount of 10% by weight based on the total weight of the composition, in an amount of 15% by weight based on the total weight of the composition, in an amount of 20% by weight based on the total weight of the composition, in an amount of 25% by weight based on the total weight of the composition, in an amount of 30% by weight based on the total weight of the composition.

Preferably, the composition according to the invention further comprises at least one additive chosen from the group consisting of lubricants, surfactants, antioxidants, chelants, the derivatives thereof and the mixtures thereof.

By way of example, the following compounds can be used, alone or as a mixture, as lubricants in a composition according to the invention: glycerol dibhenate, talc, silica, stearic acid, boric acid, waxes, sodium oleate, sodium acetate, magnesium stearate, calcium stearate, sodium stearate, sodium benzoate, sodium lauryl sulfate, glycerol distearate, glycerol palmitostearate, microcrystalline cellulose or even polyoxyl-8-glycerides.

By way of example, the following compounds can be used, alone or as a mixture, as surfactants in a composition according to the invention: Pluronic®, Span®, Cremophor®, polysorbates (Tween®, . . . ), vitamin E TPGS and sodium ducosate.

By way of example, the following compounds can be used, alone or as a mixture, as antioxidants and/or chelants in a composition according to the invention: butylated hydroxytoluene, butylated hydroxyanisiole, EDTA, citric acid and vitamin E.

Advantageously, the composition according to the invention further comprises at least one additional compound of polyphenol type chosen from the group consisting of phenolic acids, stilbenes, phenolic alcohols, lignans, flavonoids, the derivatives thereof and the mixtures thereof. In particular, the glycosylated and aglycone forms of the polyphenols are envisaged as an additional active ingredient according to the present invention. More particularly, within the meaning of the present invention, the term “polyphenol” designates both polyphenols of natural origin and synthetic polyphenols but also all polyphenol derivatives.

By way of example, within the meaning of the present invention, the derivatives of hydroxybenzoic acid (gallic acid, tannic acid, . . . ) and the derivatives of hydroxycinamic acid (curcumin, coumaric acid, caffeic acid, ferulic acid, . . . ) may be mentioned as phenolic acids.

By way of example, within the meaning of the present invention, resveratrol, sirtinol, piceatannol or else polydatin may be mentioned as stilbenes.

By way of example, within the meaning of the present invention, flavanoles (quercetin, myricetin, kaempferol, isorhamnetin, morine, rutin, tiliroside, trihydroxyethylrutin, fisetin, . . . ), flavones (apigenin, luteolin, baicalein, chrysin, diosmin, nobiletin, tangeretin, wogonin, aminogenistein, . . . ), flavanones (bavachine, 8-isopentenylnaringenin, isoxanthohumole, naringenin, eriodictyole, hesperetin, silybin, taxifolin, . . . ), isoflavones (genistein, daidzein, daidzin, formonetin, genistin, neobavaoflavone, pueranin, . . . ), antocianidins (cianidin, pelargonidin, delphinidin, petunidin, malvidin, . . . ) and flavanols (cathechins, gallocatechin, epigallocatechingallate, . . . ) may be mentioned as flavonoids.

According to the invention, said at least one additional active ingredient of polyphenol type constitutes an inhibitor/modulator of the efflux pumps including P-gp.

Preferentially, the composition according to the invention further comprises at least one inhibitor and/or a modulator of the activity of the P-gp.

Preferably, the composition according to the invention is packaged in the form of pellets, flakes, granules, powders, effervescent or non-effervescent tablets, injectable or non-injectable solutions, suspensions, gels, ointments or even in any other suitable form allowing administration to an animal or to a human being.

Other embodiments of a composition according to the invention are indicated in the appended claims.

An object of the invention is also a method for manufacturing, in particular a method for manufacturing by thermoforming, a composition in the form of a friable/brittle thermoformed extrudate according to the invention, characterized in that it comprises the following steps:
a) a step of simultaneous or delayed supplying at least one natural or synthetic non-euphoric cannabinoid and at least one natural or synthetic carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibres, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof, to be fed into an extruder,
b) a step of mixing, in said extruder, said at least one natural or synthetic non-euphoric cannabinoid and said at least one natural or synthetic carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibres, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof, to form a mixture, and
c) a step of hot melt extruding said mixture obtained in step b) in said extruder to obtain a friable/brittle thermoformed extrudate, in particular to obtain a friable/brittle thermoformed extrudate consisting of a solid dispersion, more particularly to obtain a thermoformed extrudate consisting of a solid dispersion consisting of a glassy structure comprising a molecular mixture of said at least one non-euphoric cannabinoid and of said at least one carrier.

Such a method according to the invention gives rise to a composition in the form of a friable/brittle thermoformed extrudate, that is to say obtained by thermoforming and more particularly by hot melt extrusion, in which said at least one natural or synthetic non-euphoric cannabinoid as active ingredient preferentially comprises at least one first amorphous phase and optionally one second crystalline phase dispersed within said at least one carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibres, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof.

This composition according to the invention has a significantly higher solubility and/or dispersion in aqueous phase (aqueous medium) of said at least one natural or synthetic non-euphoric cannabinoid and simultaneously a significantly increased bioavailability of said at least one natural or synthetic non-euphoric cannabinoid relative to solubilities and/or to dispersions and to bioavailabilities of these compounds for the current compositions. It has been shown that the composition according to the invention is in the form of a friable/brittle thermoformed extrudate in which said at least one natural or synthetic non-euphoric cannabinoid (active ingredient) preferentially comprising at least one first amorphous phase and optionally one second crystalline phase is dispersed within said at least one carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibres, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof.

It has also been shown, in the context of the present invention, that said at least one natural or synthetic non-euphoric cannabinoid is not degraded even during the method for manufacturing by thermoforming the composition in the form of a friable/brittle thermoformed extrudate which nevertheless implies the submission of said at least one natural or synthetic non-euphoric cannabinoid to high temperatures (HME). Moreover, it has also been demonstrated that said at least one natural or synthetic non-euphoric cannabinoid in a composition in the form of a friable/brittle thermoformed extrudate according to the invention is homogeneously spread (distributed) therein.

More particularly, the Hot Melt Extrusion HME carried out according to the thermoforming manufacturing method according to the invention gives rise to a melting of the active ingredient (said at least one natural or synthetic non-euphoric cannabinoid) and/or of said at least one carrier at a temperature greater than or equal to their melting point.

However, according to certain embodiments of a composition according to the invention, this melting of the active ingredient and of the carrier can take place at a temperature below their melting point. This is the case, for example, if the composition according to the invention comprises a plasticizer or if the active ingredient itself has plasticizing properties. Such a melting of the active ingredient and/or of the carrier gives rise to a solid dispersion in which the active ingredient (said at least one natural or synthetic non-euphoric cannabinoid) preferentially comprising at least one first amorphous phase and optionally one second crystalline phase is dispersed/spread/distributed homogeneously within said at least one carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof.

Advantageously, according to the method according to the invention, step c) of hot melt extrusion is a step according to which said non-euphoric cannabinoid and/or said at least one carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof, undergoes/undergo a change of state of the material, said at least one non-euphoric cannabinoid and/or said at least one carrier changing from a first state of the material to a second state of the material, which second state of the material implying in particular that said at least one non-euphoric cannabinoid and/or said at least one carrier is/are at least partially in amorphous form.

Advantageously, the method according to the invention comprises a preliminary step of premixing said at least one natural or synthetic non-euphoric cannabinoid and said at least one carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof, such so as to form a premixture intended to be fed into the extruder.

Preferably, according to the method according to the invention, said hot melt extrusion step is carried out at an extrusion temperature or thermoforming temperature comprised between 20 and 180° C., preferably at a temperature comprised between 40 and 115° C., preferentially at a temperature comprised between 80 and 90° C., preferably at a temperature equal to 115° C., preferably at a temperature equal to 110° C., preferably at a temperature equal to 100° C., preferably at a temperature equal to 90° C., preferably at a temperature equal to 80° C.

Advantageously, according to the method according to the invention, said hot melt extrusion step is carried out at a rotation speed of an extrusion screw comprised between 20 and 900 rpm, preferably comprised between 50 and 300 rpm, preferably comprised between 100 and 250 rpm, preferentially equal to 250 rpm, preferentially equal to 200 rpm.

Preferentially, the method according to the invention comprises an additional step of cooling at the outlet of the extruder.

Advantageously, the method according to the invention comprises an additional step of treating the friable/brittle thermoformed extrudate at the outlet of the extruder, for example cutting at a pelletiser and/or grinding and/or crushing and/or powdering (in powder form) said friable/brittle thermoformed extrudate.

Other embodiments of the method according to the invention are indicated in the appended claims.

The present invention also relates to a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, obtained according to the method according to the invention, said composition comprising at least one natural or synthetic non-euphoric cannabinoid as active ingredient and at least one carrier selected from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof, said at least one natural or synthetic non-euphoric cannabinoid preferentially comprising at least one first amorphous phase and optionally one second phase crystalline.

In other words, the present invention also relates to a composition in the form of a friable/brittle thermoformed extrudate, optionally packaged in pellet, flake, granule, powder, effervescent or non-effervescent tablet, injectable or non-injectable solution, suspension, gel, ointment or even in any other suitable form allowing administration to an animal or to a human being, obtained by Hot Melt Extrusion, HME, said composition comprising at least one natural or synthetic non-euphoric cannabinoid as active ingredient and at least one carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof, said at least one natural or synthetic non-euphoric cannabinoid preferentially comprising at least one first amorphous phase and optionally one second crystalline phase.

The present invention also relates to a use of a composition according to the invention as a food supplement and/or as a cosmetic product and/or as a medicament for human or veterinary use.

In particular, the present invention relates to a composition for use in the preventive and/or curative treatment, in human being and/or in animal, of pathologies related to the cardiovascular system (ischemia, metabolic disorders, . . . ), pathologies related to the gastrointestinal system (nausea, vomiting, diarrhea, digestive inflammations, . . . ), pathologies related to the premature aging of cells, pathologies related to the endocrine system (hyperglycaemia, . . . ), pathologies related to the immune system (inflammatory diseases, rheumatoid arthritis, multiple sclerosis, osteoarthritis, fibromyalgia, . . . ), pathologies related to the central nervous system (pain, migraine, epilepsy, Parkinson's disease, Alzheimer's disease, anxiety, depression, . . . ), sleep disorders, skin diseases, diseases due to the presence of microorganisms, cancers (anti-tumoral, . . . ), eye diseases (glaucoma, . . . ), diseases of the respiratory system (asthma, . . . ) and in the preventive and/or curative treatment of diabetes.

More particularly, the present invention relates to a composition for use in the preventive and/or curative treatment, in human being and/or in animal, of diseases related to neuropsychiatric disorders, pain, neurodegenerative diseases, inflammatory diseases, dermatitis and skin diseases or even irritable bowel (IBS).

More particularly, the present invention relates to a composition used, in human being and/or in animal, as an antioxidant, anti-inflammatory, anticonvulsant, antiemetic, anxiolytic, hypnotic or even as an antipsychotic.

A composition according to the invention preferentially has anti-inflammatory, neuroprotective, anticonvulsant, anxyolitic, antidepression, antipsychotic, antithrombotic, antitumor, antidiabetic and/or immunoregulatory properties.

Other forms of use of a composition according to the invention are indicated in the appended claims.

Other characteristics, details and advantages of the invention will emerge from the examples given below, not by way of limitation and with reference to the appended figures.

FIGS. 1 to 3 are graphs illustrating the rate of dispersion and solubilization of a non-euphoric cannabinoid, in this case the rate of dispersion and solubilization of cannabidiol over time, starting from examples of compositions according to the invention.

### EXAMPLES

### Example 1: Method for Manufacturing by Thermoforming Compositions According to the Invention in the Form of Friable/Brittle Thermoformed Extrudates

Thermoformed compositions according to the invention comprising at least one non-euphoric cannabinoid, such as those forming the subject of Example 2 below, were obtained according to the following method also forming the subject of the present invention:
a) a step of premixing at least one non-euphoric cannabinoid in the crystalline state in powder form and at least one carrier chosen from the group consisting of amino acids, natural or synthetic peptides and polypeptides, natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof, natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, urea, the derivatives thereof and the mixtures thereof;
b) a step of supplying said premixture formed in step a) to be fed into a Process 11 Hygienic type extruder of Thermo-Fischer®;
c) a step of mixing, in said extruder, said premixture to obtain a mixture;
d) a step of hot melt extrusion thermoforming said mixture obtained in step c) in said extruder to obtain a friable/brittle thermoformed extrudate, the hot melt extrusion step being carried out at a rotation speed of an extrusion screw of 200 rpm and at a temperature comprised between 20 and 80° C.,
e) a step of cooling at the outlet of the extruder said friable/brittle thermoformed extrudate obtained in step d); and
f) a step of cutting/grinding, at a grinder, the cooled friable/brittle thermoformed extrudate obtained in step e) in such a way to to obtain a homogeneous powder, that is to say a thermoformed extrudate packaged in powder (in powder form).

The thermoforming temperature at which the hot melt extrusion step is carried out is determined by the type of constituents used, in particular according to the type of carrier and/or plasticizer used, which the person skilled in the art is able to determine. Moreover, a person skilled in the art, in particular according to the type of extruder employed and in accordance with the general principle of the hot melt extrusion (HME), is able to define possible temperature stages in different zones along the extrusion screw(s) so that there is a gradual increase in the temperature within the material transported by the extrusion screw(s), this in a advancing direction of the material within the extruder. Typically, between zones defined along the extrusion screw(s), temperature differences in the range of 0 to 40° C. are observed. For example, in the context of the present invention, the compositions tested below were obtained in a Process 11 Hygienic type extruder of Thermo-Fischer® having 8 temperature zones which are as follows in a advancing direction of the material moving at a speed of 200 rpm: 20° C.-40° C.-60° C.-60° C.-80° C.-80° C.-80° C. and 80° C.

### Example 2: Dispersion and Solubility Test of Thermoformed Compositions According to the Invention

Thermoformed compositions, obtained according to the manufacturing method described in example 1, were tested in terms of dispersion and solubility of cannabidiol. This dispersion and this solubility were measured over time starting from thermoformed extrudates obtained according to the invention. As indicated above, the thermoformed extrudates are in the form of a homogeneous powder (ground material) in which cannabidiol (a non-euphoric cannabinoid) preferentially comprises at least one amorphous phase.

The dispersion and solubility test was carried out with a vane dissolution apparatus starting with approximately 360 mg of thermoformed extrudate, at a temperature of 37° C. under stirring at 50 rpm in 900 ml of a 0.1N HCl dissolution medium. This dispersion and solubility test was carried out according to the recommendations of the pharmacopoeia Ph.Eur.9.0 (Recommendations on Dissolution Testing). At determined times (after 15 min, after 30 min, after 1 h and after 2 h), a sample of 1 ml is taken to carry out the dispersion and solubility test.

In order to perform the dispersion and solubility test, the sample is diluted in an appropriate solvent (methanol) then filtered through a filter (PET, pore size of 0.45 μm, Macherey Nagel) before HPLC analysis (Nucleodur 100-5 EC C18 125/4 column (Macherey-Nagel): mobile phase: 85%—A (Methanol) and 15% B (Water); flow rate: 1 ml/min, loop: 10 μl, t°=25° C.; 220 nm).

The thermoformed compositions according to the invention listed in Table 1 were formulated according to the method of the invention and tested in terms of dispersion and solubility over time according to the principle indicated above. Cannabidiol (CBD) in native crystalline form and in powder form (native CBD) was used as a control. The amounts mentioned in Table 1 are percentages by weight of the compounds used (subjected to the method according to the invention) based on the total weight of the composition.

TABLE 1

| | Cannabidiol | | | |
|---|---|---|---|---|
| | (CBD) (1) | Protein (2) | Saccharide (3) | Glycerol (4) |
| Compo 1 | 20 | 70 | 0 | 10 |
| Compo 2 | 20 | 0 | 70 | 10 |

(1) Cannabidiol (Shanghai Freemen)
(2) Bovine collagen 5000 Da (Rousselot)
(3) Modified Starch Cleargum ®CO01 (Roquette)
(4) Glycerol (Brenntag)

The results of the dispersion and solubility tests obtained are shown in FIG. 1. As can be seen, the dispersion+the solubility measured for the CBD starting from compositions in the form of thermoformed extrudates according to the invention (Compo 1 and Compo 2) is clearly higher relative to the control (native CBD).

### Example 3: Dispersion and Solubility Test of Thermoformed Compositions According to the Invention

Thermoformed compositions, obtained according to the manufacturing method described in Example 1, were tested in terms of dispersion and solubility of cannabidiol. This dispersion and this solubility were measured over time starting from thermoformed extrudates obtained according to the invention. As indicated above, the thermoformed extrudates are in the form of a homogeneous powder (ground material) in which cannabidiol (a non-euphoric cannabinoid) preferentially comprises at least one amorphous phase.

The dispersion and solubility test was carried out as indicated in Example 2.

The thermoformed compositions according to the invention listed in Table 2 were formulated according to the method of the invention and tested in terms of dispersion and solubility over time according to the principle indicated above. Cannabidiol (CBD) in native crystalline form and in powder form (native CBD) was used as a control. The amounts mentioned in Table 2 are percentages by weight of the compounds used (subjected to the method according to the invention) based on the total weight of the composition.

TABLE 2

| | Cannabidiol | | |
|---|---|---|---|
| | (CBD) (1) | Protein (2) | Glycerol (3) |
| Compo 1 | 20 | 70 | 10 |
| Compo 2 | 25 | 70 | 5 |
| Compo 3 | 30 | 70 | 0 |

(1) Cannabidiol (Shanghai Freemen)
(2) Collagen Peptan B 5000 HD (Ingrizo)
(3) Glycerol (Brenntag)

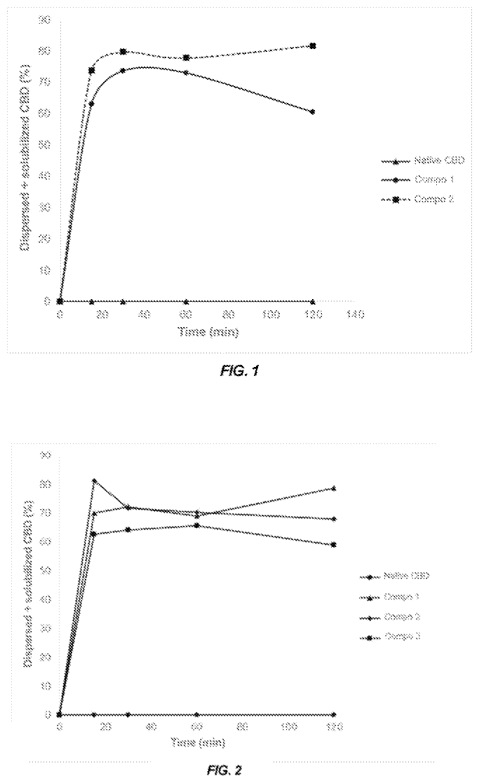

The results of the dispersion and solubility tests obtained are shown in FIG. 2. As can be seen, the dispersion+the solubility measured for the CBD starting from compositions in the form of thermoformed extrudates according to the invention (Compo 1, Compo 2 and Compo 3) is significantly higher relative to the control (native CBD).

### Example 4: Dispersion and Solubility Test of Thermoformed Compositions According to the Invention

Thermoformed compositions, obtained according to the manufacturing method described in Example 1, were tested in terms of dispersion and solubility of cannabidiol. This dispersion and this solubility were measured over time starting from thermoformed extrudates obtained according to the invention. As indicated above, the thermoformed extrudates are in the form of a homogeneous powder (ground material) in which cannabidiol (a non-euphoric cannabinoid) preferentially comprises at least one amorphous phase.

The dispersion and solubility test was carried out as indicated in Example 2.

The thermoformed compositions according to the invention listed in Table 3 were formulated according to the method of the invention and tested in terms of dispersion and solubility over time according to the principle indicated above. Cannabidiol (CBD) in native crystalline form and in powder form (native CBD) was used as a control. The amounts mentioned in Table 3 are percentages by weight of the compounds used (subjected to the method according to the invention) based on the total weight of the composition.

TABLE 3

| | Cannabidiol | | |
|---|---|---|---|
| | (CBD) (1) | Saccharide | Glycerol (6) |
| Compo 1 | 20 | 70 (2) | 10 |
| Compo 2 | 20 | 70 (3) | 10 |
| Compo 3 | 20 | 70 (4) | 10 |
| Compo 4 | 20 | 70 (5) | 10 |

(1) Cannabidiol (Shanghai Freemen)
(2) Dextrin Tackidex ® C760 (Roquette)
(3) Modified Starch Cleargum ® CB95 (Roquette)
(4) Modified Starch Cleargum ® MG85 (Roquette)
(5) Modified Starch Cleargum ® LG7015 (Roquette)
(6) Glycerol (Brenntag)

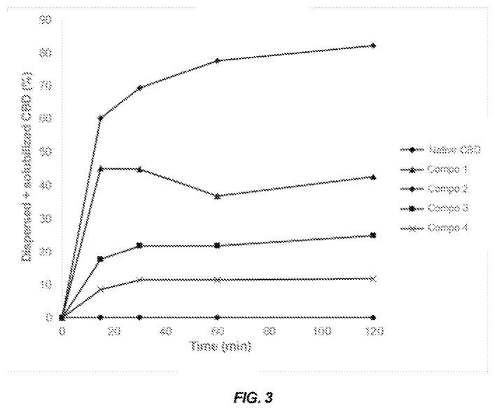

The results of the dispersion and solubility tests obtained are shown in FIG. 3. As can be seen, the dispersion+the solubility measured for the CBD starting from compositions in the form of thermoformed extrudates according to the invention (Compo 1, Compo 2, Compo 3 and Compo 4) is always higher relative to the control (native CBD).

The present invention has been described in relation to the specific embodiments, which have a purely illustrative value and should not be considered as limitative. In general, it will appear obvious to the person skilled in the art that the present invention is not limited to the examples illustrated and/or described above.

The use of the verbs “comprise”, “include”, or any other variant, as well as the conjugations thereof, can in no way exclude the presence of elements other than those mentioned.

The use of the indefinite article “a”, “an”, or of the definite article “the”, to introduce an element does not exclude the presence of a plurality of these elements.

1-16. (canceled)

17. A composition in the form of a friable/brittle thermoformed extrudate, packaged in at least one of a pellet, a flake, a granule, a powder, an effervescent tablet, a non-effervescent tablet, an injectable solution, a non-injectable solution, a suspension, a gel, or an ointment for allowing administration to an animal or to a human being, the composition comprising:
at least one natural or synthetic non-euphoric cannabinoid; and
at least one natural or synthetic carrier chosen from the group consisting of:
amino acids,
natural or synthetic peptides and polypeptides,
natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof,
natural or synthetic saccharides selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof,
natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, and
urea, the derivatives thereof and the mixtures thereof.

18. The composition according to claim 17, where the friable/brittle thermoformed extrudate includes a thermoformed mixture of:
at least one natural or synthetic non-euphoric cannabinoid, and
of at least one natural or synthetic carrier chosen from the group consisting of:
amino acids,
natural or synthetic peptides and polypeptides,
natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof,
natural or synthetic saccharides selected from calcium alginate, sodium alginate, ammonium alginate, propylene glycol alginate, starches, pregelatinized starches, modified starches, soluble or insoluble fibers, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof,
natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, and
urea, the derivatives thereof and the mixtures thereof.

19. The composition according to claim 17, wherein the at least one natural or synthetic non-euphoric cannabinoid is chosen from the group consisting of cannabidiol (CBD), cannabigerol (CBG), cannabichromene (CBC), cannabidivarin (CBDV), cannabidiolic acid (CBDA), cannabigerovarin (CBGV), cannabichromenic acid (CBCA), cannabiorcichromenic acid, 8-chloro-cannabiorcichromenic acid, cannachromevarinic acid (CBCVA), lacannabielsoin (CBE), cannabicyclol (CBL), perrottetinene, perrottittenic acid, lunularic acid, vittatin, helichrysum CBG, amorfrutins (A, B, C and D), machaeridiols (A, B, C), daurichromenic acid, anthopogocyclolic acid, anthopogochromenic acid, geranyl-orsellinic acid, confluentine, griffolic acid, rubiginosin A, the derivatives thereof and the mixtures thereof.

20. The composition according to claim 17, wherein at least one of the collagens, the collagen hydrolysates, or the gelatins have a molecular weight between 50 Da and 300000 Da.

21. The composition according to claim 20, wherein the molecular weight is between 1200 and 1000 Da.

22. The composition according to claim 17, further including at least one plasticizer.

23. The composition according to claim 17, further including at least one additive chosen from the group consisting of lubricants, surfactants, antioxidants, chelants, the derivatives thereof and the mixtures thereof.

24. The composition according to claim 17, further including at least one polyphenol type compound chosen from the group consisting of phenolic acids, stilbenes, phenolic alcohols, lignans, flavonoids, the derivatives thereof and the mixtures thereof.

25. A method of treating one or more pathologies related to at least one of a cardiovascular system, a gastrointestinal system, a premature aging of cells, an endocrine system, an immune system, a central nervous system, a sleep disorder, a skin disease, a disease due to a presence of a microorganism, a cancer, an eye disease, a disease of a respiratory system, or diabetes, the method comprising administering the composition of claim 17 to a subject.

26. The method according to claim 25, wherein the one or more pathologies including at least one of ischemia, a metabolic disorder, nausea, vomiting, diarrhea, digestive inflammations, hyperglycaemia, an inflammatory disease, rheumatoid arthritis, multiple sclerosis, osteoarthritis, fibromyalgia, pain, migraine, epilepsy, Parkinson's disease, Alzheimer's disease, anxiety, depression, a tumor, glaucoma, or asthma.

27. A thermoforming method for manufacturing a composition in the form of a friable/brittle thermoformed extrudate, the method comprising:
a) simultaneous or delayed supplying into an extruder of:
at least one natural or synthetic non-euphoric cannabinoid, and
at least one natural or synthetic carrier chosen from the group consisting of:
amino acids,
natural or synthetic peptides and polypeptides,
natural or synthetic proteins selected from glycoproteins, collagens, collagen hydrolysates, gelatins, pea proteins, soybean proteins, wheat proteins, pumpkin proteins, nut proteins, rice proteins, hazelnut proteins, milk proteins, green pea proteins, lentil proteins, almond proteins, pistachio proteins, chia proteins, hemp proteins, flax proteins, butternut proteins, sesame proteins, poppy proteins, lupine proteins, sunflower proteins, oat proteins, millet proteins, barley proteins, rye proteins, the derivatives thereof and the mixtures thereof, natural or synthetic saccharides selected from calcium alginate, sodium alginate, ammonium alginate, propylene glycol alginate, starches, pregelatinized starches, modified starches, soluble or insoluble fibres, carrageenans, pectin, hydroxypropyl cellulose (HPC), glycogen, β-glucan, amylopectin, amylose, dextrins, cyclodextrins, maltodextrin, isomaltose, isomalt, xylan, pullulan, agar-agar, mannans, fucoidan, xanthan gum, guar gum, mastic gum, gum arabic, acacia gum, gum tragacanth, gum ghatti, karaya gum, Konjac gum, fenugreek gum, cashew or cashew nut gum, tara gum, carob bean gum, cassia gum, benzoin gum, gum cassia, guaiac gum, chitosan, chitin, levan, neoserine, hyaluronic acid, hyaluronates, chondroitin sulfate, dermatan sulfate, keratan sulfate, raffinose, rhamminose, mannose, glucose, rhamnose, sucrose, stachyose, verbascose, trehalose, lactose, lactulose, maltose, erythritol, mannitol, sorbitol, xylitol, the derivatives thereof and the mixtures thereof,
natural or synthetic lipids selected from beeswax, candelilla wax, rice bran wax, carnauba wax, microcrystalline waxes, animal or vegetable oils, hydrogenated animal or vegetable oils, phospholipids, ceramides, fatty acid esters, the derivatives thereof and the mixtures thereof, and
urea, the derivatives thereof and the mixtures thereof;
b) mixing, in the extruder, the at least one natural or synthetic non-euphoric cannabinoid and the at least one natural or synthetic carrier to form a mixture; and
c) hot melt extruding the mixture in the extruder to obtain the friable/brittle thermoformed extrudate.

28. The method according to claim 27, wherein the friable/brittle thermoformed extrudate includes a solid dispersion.

29. The method according to claim 27, wherein the friable/brittle thermoformed extrudate includes a glassy structure including a molecular mixture of the at least one non-euphoric cannabinoid and of the at least one carrier.

30. The method according to claim 27, further including premixing the at least one natural or synthetic non-euphoric cannabinoid and the at least one natural or synthetic carrier to form a premixture prior to supplying the premixture into the extruder.

31. The method according to claim 27, wherein the hot melt extruding includes an extrusion temperature between 20 and 180° C.

32. The method according to claim 31, wherein the extrusion temperature is about 80° C.

33. The method according to claim 27, wherein the hot melt extruding includes a rotation speed of an extrusion screw between 20 and 900 rpm.

34. The method according to claim 33, wherein the rotation speed is 200 rpm.

35. The method according to claim 27, further including cooling at an outlet of the extruder.

36. The method according to claim 27, further including treating the friable/brittle thermoformed extrudate at an outlet of the extruder by at least one of cutting the extrudate at a pelletizer, grinding the extrudate, crushing the extrudate, or powdering the extrudate.

37. A composition in the form of a friable/brittle thermoformed extrudate packaged in at least one of a pellet, a flake, a granule, a powder, an effervescent table, a non-effervescent tablet, an injectable solution, a non-injectable solution, a suspension, a gel, or an ointment for administration to an animal or to a human being, obtained according to the method of claim 27.
